(19) United States
(12) Patent Application Publication
(10) Pub. No.: US 2026/0167627 A1
(43) Pub. Date: Jun. 18, 2026

(54) NEW SOLID FORMS OF (3R)-N-[2-CYANO-4-FLUORO-3-(3-METHYL-4-OXO-QUINAZOLIN-6-YL)OXY-PHENYL]-3-FLUORO-PYRROLIDINE-1-SULFONAMIDE

(71) Applicant: Hoffmann-La Roche Inc., Little Falls, NJ (US)

(72) Inventors: Giacomo Marco LARI, Basel (CH); Sabine SCHWARZ, Bad Saeckingen (DE); Valentin Andreas STADELMANN, Bettingen (CH)

(73) Assignee: Hoffmann-La Roche Inc., Little Falls, NJ (US)

(21) Appl. No.: 19/420,124

(22) Filed: Dec. 15, 2025

Related U.S. Application Data
(63) Continuation of application No. PCT/EP2024/068524, filed on Jul. 2, 2024.

(30) Foreign Application Priority Data
Jul. 4, 2023 (EP) 23183396.3

(51) Int. Cl. C07D 403/12; A61K 9/02; A61K 9/20; A61K 9/48; A61K 31/506; A61K 47/12; A61K 47/34

(52) U.S. Cl. CPC C07D 403/12; A61K 9/02; A61K 9/2013; A61K 9/2018; A61K 9/2054; A61K 9/4825; A61K 9/485; A61K 9/4858; A61K 9/4866; A61K 31/506; A61K 47/12; A61K 47/34

(57) ABSTRACT

The present invention provides solid forms of (3R)—N-[2-cyano-4-fluoro-3-(3-methyl-4-oxo-quinazolin-6-yl)oxy-phenyl]-3-fluoro-pyrrolidine-1-sulfonamide and solvates thereof, as well as therapeutic uses thereof and pharmaceutical composition comprising them.

### FIELD OF THE INVENTION

The present invention provides new solid forms of (3R)—N-[2-cyano-4-fluoro-3-(3-methyl-4-oxo-quinazolin-6-yl)oxy-phenyl]-3-fluoro-pyrrolidine-1-sulfonamide, as well as therapeutic uses thereof and pharmaceutical compositions comprising said forms.

### BACKGROUND ART

The Rapidly Accelerated Fibrosarcoma (RAF) class of serine-threonine kinases comprise three members (ARAF, BRAF, RAF1) that compose the first node of the MAP kinase signalling pathway. Despite the apparent redundancy of the three RAF isoforms in signalling propagation through phosphorylation of MEK1 and 2, frequent oncogenic activating mutations are commonly found only for BRAF. In particular, substitution of V600 with glutamic acid or lysine renders the kinase highly activated with consequent hyper-stimulation of the MAPK pathway, independently from external stimulations (Cell. 2015 Jun. 18; 161 (7): 1681-1696).

Mutant BRAF is a targetable oncogenic driver and three BRAF inhibitors (vemurafenib, dabrafenib and encorafenib) reached the market up to now showing efficacy in BRAFV600E-positive melanoma. However rapid acquisition of drug resistance is almost universally observed and the duration of the therapeutic benefits for the targeted therapy remains limited.

Moreover, the developed BRAF inhibitors revealed an unexpected and “paradoxical” ability to repress MAPK signalling in BRAFV600E-driven tumours while the same inhibitors presented MAPK stimulatory activities in BRAF wild type (WT) models (N Engl J Med 2012; 366:271-273; and British Journal of Cancer volume 111, pages 640-645 (2014)).

Mechanistic studies on the RAF paradox then clarified that oncogenic BRAFV600E phosphorylates MEK 1/2 in its monomeric cytosolic form while WT BRAF and RAF1 activation requires a complex step of events including cell membrane translocation and homo and/or heterodimerization promoted by activated RAS (KRAS, NRAS, HRAS) (Nature Reviews Cancer volume 14, pages 455-467 (2014)).

The binding of inhibitors like vemurafenib, dabrafenib or encorafenib to a WT BRAF or RAFI protomer, quickly induces RAF homo and/or hetero dimerization and membrane association of the newly formed RAF dimer. In the dimeric conformation, one RAF protomer allosterically induces conformational changes of the second resulting in a kinase active status and, importantly, in a conformation unfavourable for the binding of the inhibitor. The dimer induced by drug treatment, as a result, promotes MEK phosphorylation by the catalysis operated by the unbound protomer with hyperactivation of the pathway.

The RAF paradox results in two clinically relevant consequences: 1) accelerated growth of secondary tumours upon BRAFi monotherapy (mainly keratochantoma and squamous-cell carcinomas) (N Engl J Med 2012; 366:271-273) and 2) the acquisition of drug resistance in the setting of BRAFi monotherapy as well as in combinations of BRAFi+MEKi presents activation of dimer-mediated RAF signalling by genetically driven events including RAS mutations, BRAF amplifications, expression of dimeric-acting BRAF splice variants (Nature Reviews Cancer volume 14, pages 455-467 (2014)). There is thus the need for RAF inhibitors capable of breaking that paradox.

Furthermore, the currently approved classical BRAF inhibitors Vemurafenib (Mol. Pharmaceutics 2012, 9, 11, 3236-3245), Dabrafenib (J Pharmacol Ex Ther 2013, 344 (3) 655-664) and Encorafenib (Pharmacol Res. 2018; 129:414-423) all have very poor brain permerability. This is major limitation for the use of those classical BRAF inhibitors for the treatment of brain cancer or brain metastases. There is thus the need for BRAF inhibitors having improved brain permeability.

There is accordingly a need for compounds that are efficient BRAF inhibitors showing considerably less paradoxial activation of the MAPK signaling pathway while retaining high potency. Such compounds can be referred to as a paradox breaker or RAF paradox breaker, in contrast to compounds inducing the RAF paradox (and which could be referred to as paradox inducers or RAF paradox inducers). (3R)—N-[2-cyano-4-fluoro-3-(3-methyl-4-oxo-quinazolin-6-yl)oxy-phenyl]-3-fluoro-pyrrolidine-1-sulfonamide satisfies these needs, and is a paradox breaking BRAF inhibitor with favourable brain penetration properties.

Polymorphs are different crystalline forms of the same compound. Polymorphs typically have a different crystal structure due to a different packing of the molecules in the lattice. Polymorphic forms are of interest to the pharmaceutical industry and especially to those involved in the development of suitable dosage forms. If the polymorphic form is not held constant during clinical studies, the exact dosage form used or studie may not be comparable form one lot to another. It is also desirable to have processes for producing a compound with the selected polymorphic form in high purity when the compound is used in clinical studies or commercial products since any impurities may produce undesired effects (e.g. toxicity). Certain polymorphs may display may also exhibit enhanced stability or may be more readily manufactured in high purity in large quantities, and are more suitable for inclusion in pharmaceutical formulations. Certain polymorphs may display other advantageous physical properties such as lack of hygroscopic tendencies, improved solubility, and enhanced rates of dissolution due to different lattic energies.

(3R)—N-[2-cyano-4-fluoro-3-(3-methyl-4-oxo-quinazolin-6-yl)oxy-phenyl]-3-fluoro-pyrrolidine-1-sulfonamide is a pardox breaking BRAF inhibitor with favourable brain penetration properties and useful in the therapy of cancer, in particular melanoma, lung cancer and brain metastatic cancer. Accordingly, for pharmaceutical development and commercialization, there is a need to identify solid forms of (3R)—N-[2-cyano-4-fluoro-3-(3-methyl-4-oxo-quinazolin-6-yl)oxy-phenyl]-3-fluoro-pyrrolidine-1-sulfonamide having desirable properties such as high crystallinity, high purity, and favourable physical stability, chemical stability, dissolution and mechanical properties. WO2022/258584 described procedures to isolate the polymorphic solid form A, as well as amorphous (3R)—N-[2-cyano-4-fluoro-3-(3-methyl-4-oxo-quinazolin-6-yl)oxy-phenyl]-3-fluoro-pyrrolidine-1-sulfonamide. The present invention provides (3R)—N-[2-cyano-4-fluoro-3-(3-methyl-4-oxo-quinazolin-6-yl)oxy-phenyl]-3-fluoro-pyrrolidine-1-sulfonamide in a novel solid forms, namely crystalline polymorphic form B.

### DETAILED DESCRIPTION OF THE INVENTION

The present invention relates to a solid form of a compound of formula (I)

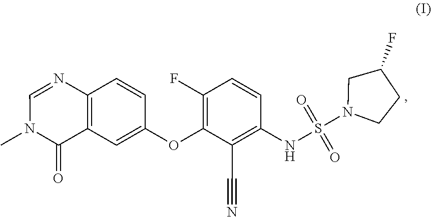 [SMILES: Cn1cnc2ccc(Oc3c(F)ccc(NS(=O)(=O)N4CC[C@@H](F)C4)c3C#N)cc2c1=O]

wherein the solid form is crystalline polymorph Form B.

The compound of formula (I) is also referred to as (3R)—N-[2-cyano-4-fluoro-3-(3-methyl-4-oxo-quinazolin-6-yl)oxy-phenyl]-3-fluoro-pyrrolidine-1-sulfonamide.

Crystalline polymorphic Form B is the thermodynamically stable form of (3R)—N-[2-cyano-4-fluoro-3-(3-methyl-4-oxo-quinazolin-6-yl)oxy-phenyl]-3-fluoro-pyrrolidine-1-sulfonamide. The crystalline polymorphic Form B is characterized by favourable physicochemical properties, such as for example improved flowability and provides enhanced processability properties making this form suitable for large-scale manufacturing of an active pharmaceutical ingredient.

The terms “pharmaceutically acceptable carrier” and “pharmaceutically acceptable auxiliary substance” refer to carriers and auxiliary substances such as diluents or excipients that are compatible with the other ingredients of the formulation.

The term “BRAF associated cancer” refers to cancers that are associated and/or caused by activating BRAF mutations. Non-limiting examples of such such a mutation include for instance BRAF V600E and V600K mutations.

The term “room temperature” refers to 18-30° C., in particular 20-25° C., more particular to 20° C.

The terms “about” and “approximately” are interchangeably and refer to a range of values that fall within 5%, greater or less than the stated reference value. More particularly “about” or “approximately” refers to ±0.2° degrees 2-theta or ±0.5° C.

The terms “substantial amounts” as used herein, can mean at least 50%, in particular at least 60%, and more particular at least 70% of the initially present amount of a specific substance in a defined fraction. For instance after a purification step, the fraction comprising a substantial amount of a specific substance will comprise at least 50%, in particular at least 60%, more particularly at least 70% of the specific substance of the initially present amount of that specific substance prior to the purification step.

“crystallization” and “recrystallization” may be used interchangeably; referring to a process that leads to a stable polymorph or crystalline form of a particular chemical compound wherein the chemical compound prior to the process can be in amorphous form, or dissolved or suspended in a solvent system. For example, the crystallization steps can be done by forming a crystal with a solvent and an anti-solvent.

“XRPD” refers the analytical method of X-Ray Powder Diffraction. The repeatability of the angular values is in the range of 2-theta±0.2°. The term “approximately” given in combination with an angular value denotes the repeatability which is in the range of 2-theta The relative XRPD peak intensity is dependent upon many factors such as structure factor, temperature factor, crystallinity, polarization factor, multiplicity, and Lorentz factor. Relative intensities may vary considerably from one measurement to another due to preferred orientation effects. According to USP 941 (US Pharmacopoeia, 37th Edition, General Chapter 941), relative intensities between two samples of the same material may vary considerably due to “preferred orientation” effects. Anisotropic materials adopting preferred orientation will lead to anisotropic distribution of properties such as modulus, strength, ductility, toughness, electrical conductivity, thermal expansion, etc., as described e.g. in Kocks U. F. et al. (Texture and Anisotropy: Preferred Orientations in Polycrystals and Their Effect on Materials Properties, Cambridge University Press, 2000). In XRPD but also Raman spectroscopy, preferred orientations cause a change in the intensity distribution. Preferred orientation effects are particularly pronounced with crystalline APIs of relatively large particle size.

“characteristic peak” refers to the presence of the powder X-ray diffraction peak definitively identifies the (3R)—N-[2-cyano-4-fluoro-3-(3-methyl-4-oxo-quinazolin-6-yl)oxy-phenyl]-3-fluoro-pyrrolidine-1-sulfonamide as the referenced crystalline form (Form B). Typically, the powder X-ray diffraction analysis is conducted at ambient conditions in transmission geometry with a STOE STADI P diffractometer (Cu Kα₁ radiation, primary monochromator, silicon strip detector, angular range 3 to 42 degrees two-theta, approximately 30 minutes total measurement time). The samples (approximately 10 to 50 mg) are prepared between thin polymer films and are analyzed without further processing (e.g. grinding or sieving) of the substance.

“Polymorph” refers to crystalline forms having the same chemical composition but different spatial arrangements of the molecules, atoms, and/or ions forming the crystal. In general, reference throughout this specification will be to a polymorphic form of (3R)—N-[2-cyano-4-fluoro-3-(3-methyl-4-oxo-quinazolin-6-yl)oxy-phenyl]-3-fluoro-pyrrolidine-1-sulfonamide. The term “polymorphic form” as used herein may or may not include other crystalline solid state molecular forms including hydrates (e.g. bound water present in the crystalline structure) of the same compound. Polymorphs typically have a different crystal structure due to a different packing of the molecules in the lattice. This results in a different crystal symmetry and/or unit cell parameters which directly influences its physical properties such as the X-ray diffraction characteristics of crystals or powder.

“Amorphous” refers to solid materials that lack the long-range order that is characteristic of a crystalline solid.

The term “solvate” refers herein to a molecular complex comprising a compound of formula (I) and a stoichiometric or non-stoichiometric amount of one or more solvent molecules (e. g., ethanol). “Hydrate” refers herein to a solvate comprising a compound of formula (I) and a stoichiometric or non-stoichiometric amount of water.

The terms “pharmaceutically acceptable excipient”, pharmaceutically acceptable carrier” and “therapeutically inert excipient” can be used interchangeably and denote any pharmaceutically acceptable ingredient in a pharmaceutical composition having no therapeutic activity and being non-toxic to the subject administered, such as disintegrators, binders, fillers, solvents, buffers, tonicity agents, stabilizers, antioxidants, surfactants, carriers, diluents or lubricants used in formulating pharmaceutical products.

The term “pharmaceutical composition” encompasses a product comprising specified ingredients in pre-determined amounts or proportions, as well as any product that results, directly or indirectly, from combining specified ingredients in specified amounts. Particularly it encompasses a product comprising one or more active ingredients, and an optional carrier comprising inert ingredients, as well as any product that results, directly or indirectly, from combination, complexation or aggregation of any two or more of the ingredients, or from dissociation of one or more of the ingredients, or from other types of reactions or interactions of one or more of the ingredients.

The terms “pharmaceutically acceptable carrier” and “pharmaceutically acceptable auxiliary substance” refer to carriers and auxiliary substances such as diluents or excipients that are compatible with the other ingredients of the formulation.

“Therapeutically effective amount” means an amount that is effective to prevent, alleviate or ameliorate symptoms of disease or prolong the survival of the subject being treated.

The term “substantially pure” when used in reference to a solid form (3R)—N-[2-cyano-4-fluoro-3-(3-methyl-4-oxo-quinazolin-6-yl)oxy-phenyl]-3-fluoro-pyrrolidine-1-sulfonamide refers to said polymorph being >90% pure. The solid form of (3R)—N-[2-cyano-4-fluoro-3-(3-methyl-4-oxo-quinazolin-6-yl)oxy-phenyl]-3-fluoro-pyrrolidine-1-sulfonamide does not contain more than 10% of any other compound, in particular does not contain more than 10% of any other solid form of (3R)—N-[2-cyano-4-fluoro-3-(3-methyl-4-oxo-quinazolin-6-yl)oxy-phenyl]-3-fluoro-pyrrolidine-1-sulfonamide.

More particular, the term “substantially pure” when used in reference to a solid form of (3R)—N-[2-cyano-4-fluoro-3-(3-methyl-4-oxo-quinazolin-6-yl)oxy-phenyl]-3-fluoro-pyrrolidine-1-sulfonamide refers to said solid being >95% pure. The solid form of (3R)—N-[2-cyano-4-fluoro-3-(3-methyl-4-oxo-quinazolin-6-yl)oxy-phenyl]-3-fluoro-pyrrolidine-1-sulfonamide does not contain more than 5% of any other compound, in particular does not contain more than 5% of any other solid form (3R)—N-[2-cyano-4-fluoro-3-(3-methyl-4-oxo-quinazolin-6-yl)oxy-phenyl]-3-fluoro-pyrrolidine-1-sulfonamide.

Even more particular, the term “substantially pure” when used in reference to a solid form of (3R)—N-[2-cyano-4-fluoro-3-(3-methyl-4-oxo-quinazolin-6-yl)oxy-phenyl]-3-fluoro-pyrrolidine-1-sulfonamide refers to said solid form being >97% pure. The solid form of (3R)—N-[2-cyano-4-fluoro-3-(3-methyl-4-oxo-quinazolin-6-yl)oxy-phenyl]-3-fluoro-pyrrolidine-1-sulfonamide does not contain more than 3% of any other compound, in particular does not contain more than 3% of any other solid form of (3R)—N-[2-cyano-4-fluoro-3-(3-methyl-4-oxo-quinazolin-6-yl)oxy-phenyl]-3-fluoro-pyrrolidine-1-sulfonamide.

Most particular, the term “substantially pure” when used in reference to a solid form of (3R)—N-[2-cyano-4-fluoro-3-(3-methyl-4-oxo-quinazolin-6-yl)oxy-phenyl]-3-fluoro-pyrrolidine-1-sulfonamide refers to said polymorph being >99% pure. The solid form of (3R)—N-[2-cyano-4-fluoro-3-(3-methyl-4-oxo-quinazolin-6-yl)oxy-phenyl]-3-fluoro-pyrrolidine-1-sulfonamide does not contain more than 1% of any other compound, in particular does not contain more than 1% of any other solid form of (3R)—N-[2-cyano-4-fluoro-3-(3-methyl-4-oxo-quinazolin-6-yl)oxy-phenyl]-3-fluoro-pyrrolidine-1-sulfonamide.

Most particular, the term “substantially pure” when used in reference to a solid form of (3R)—N-[2-cyano-4-fluoro-3-(3-methyl-4-oxo-quinazolin-6-yl)oxy-phenyl]-3-fluoro-pyrrolidine-1-sulfonamide refers to said polymorph being >99.5% pure. The solid form of (3R)—N-[2-cyano-4-fluoro-3-(3-methyl-4-oxo-quinazolin-6-yl)oxy-phenyl]-3-fluoro-pyrrolidine-1-sulfonamide does not contain more than 1% of any other compound, in particular does not contain more than 1% of any other solid form of (3R)—N-[2-cyano-4-fluoro-3-(3-methyl-4-oxo-quinazolin-6-yl)oxy-phenyl]-3-fluoro-pyrrolidine-1-sulfonamide.

While the present invention has been described with reference to the specific embodiments thereof, it should be understood by those skilled in the art that various changes can be made and equivalents can be substituted without departing from the true spirit and scope of the invention. In addition, many modifications can be made to adapt a particular situation, material, composition of matter, process, process step or steps, to the objective spirit and scope of the present invention. All such modifications are intended to be within the scope of the claims appended hereto. All separate embodiments can be combined.

Specific numbered aspects of the invention are:
1. A solid form of a compound of formula (I)

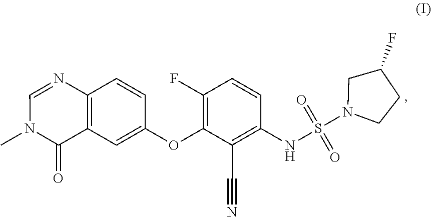 [SMILES: Cn1cnc2ccc(Oc3c(F)ccc(NS(=O)(=O)N4CC[C@@H](F)C4)c3C#N)cc2c1=O]

wherein the solid form is crystalline polymorphic Form B characterized by a X-ray powder diffraction pattern comprising a peak at an angle of diffraction at about 12.88 degrees 2-theta and at least one additional peak expressed in values of degrees 2-theta at about 10.62, 15.96, 16.82, 17.20, 20.04, 21.24, 23.78, 25.62, 25.88 or 26.90.
2. A solid form according to aspect 1, characterized by an X-ray powder diffraction pattern comprising a peak at an angle of diffraction at about 12.88 degrees 2-theta and a peak at about 10.62 degrees 2-theta.
3. A solid form according to aspect 1 or 2, characterized by an X-ray powder diffraction pattern comprising a peak at an angle of diffraction at about 12.88 degrees 2-theta and a peak at about 10.62 degrees 2-theta; wherein the pattern is further comprising at least one additional peak expressed in values of degrees 2-theta at about 15.96, 16.82, 17.20, 20.04, 21.24, 23.78, 25.62, 25.88 or 26.90.
4. A solid form according to any one of aspects 1 to 3, characterized by an X-ray powder diffraction pattern comprising at least three of the peaks at an angle of diffraction at about 10.62, 12.88, 16.82, 20.04 or 26.90 degrees 2-theta.
5. A solid form according to any one of aspects 1 to 4, characterized by an X-ray powder diffraction pattern comprising peaks at an angle of diffraction at about 10.62, 12.88, 15.96, 16.82, 17.20, 20.04, 21.24, 23.78, 25.62, 25.88 and 26.90 degrees 2-theta.
6. A solid form according to any one of aspects 1 to 5, which is further comprising a peak expressed in values of degrees 2-theta at about 8.38.
7. A solid form characterized by an X-Ray powder diffraction pattern according to any one of aspects 1 to 6, which is further comprising at least one additional peak expressed in values of degrees 2-theta at about 9.76, 10.44, 12.74, 16.68, 17.54, 18.76, 19.00, 19.40, 19.52, 20.98, 23.02, 23.30, 24.02, 26.10, 26.48, 27.24, 27.46, 28.56, 28.72, 28.94, 29.08, 29.28, 29.80 or 30.22.
8. A solid form according to any one of aspects 1 to 7, characterized by an X-ray powder diffraction pattern as shown in FIG. 1.
9. A solid form according to any one of aspects 1 to 8, characterized by having a melting point with a peak signal at about 214.2° C. to about 215.2° C., in particular with a peak signal at about 214.7° C., using differential scanning calorimetry with a heating rate of 10 K/min.
10. A solid form according to any one of aspects 1 to 9, characterized by a differential scanning calorimetry thermogram as shown in FIG. 2.
11. A solid form according to any one of aspects 1 to 9, characterized by a thermogravimetric analysis thermogram as shown in FIG. 3.
12. A solid form of a compound of formula (I)

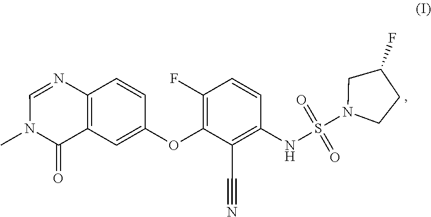 [SMILES: Cn1cnc2ccc(Oc3c(F)ccc(NS(=O)(=O)N4CC[C@@H](F)C4)c3C#N)cc2c1=O]

wherein the solid form is crystalline polymorphic Form B characterized by an IR spectrum comprising at least one peak at one of the positions 1685 cm⁻¹ (±2) cm⁻¹, 1617 cm⁻¹ (±2) cm⁻¹, 1425 cm⁻¹ (±2) cm⁻¹, 852 cm⁻¹ (±2) cm⁻¹ or 762 cm⁻¹ (±2) cm⁻¹, in particular comprising at least two peaks at positions 1685 cm⁻¹ (±2) cm⁻¹, 1617 cm⁻¹ (±2) cm⁻¹, 1425 cm⁻¹ (±2) cm⁻¹, 852 cm⁻¹ (±2) cm⁻¹ or 762 cm⁻¹ (±2) cm⁻¹, more particularly comprising the peaks at positions 1685 cm⁻¹ (±2) cm⁻¹, 1617 cm⁻¹ (±2) cm⁻¹, 1425 cm⁻¹ (±2) cm⁻¹, 852 cm⁻¹ (±2) cm⁻¹ and 762 cm⁻¹ (±2) cm⁻¹.
13. A solid form according to any one of aspects 1 to 11, further characterized by an IR spectrum according to aspect 12.
14. A solid form according to any one of aspects 1 to 11, further characterized by an IR spectrum comprising at the positions 1685 cm⁻¹ (±2) cm⁻¹
15. A solid form according to any one of aspects 1 to 14, characterized by an IR spectrum as shown in FIG. 5.
16. A solid form of a compound of formula (I)

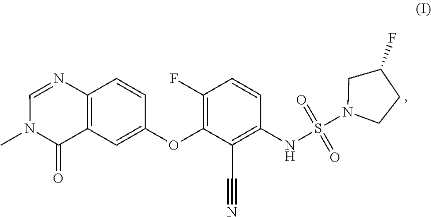 [SMILES: Cn1cnc2ccc(Oc3c(F)ccc(NS(=O)(=O)N4CC[C@@H](F)C4)c3C#N)cc2c1=O]

wherein the solid form is crystalline polymorphic Form B characterized by a Raman spectrum comprising at least one peak at one of the positions 114 (±2) cm⁻¹, 132 (±2) cm⁻¹, 167 (±2) cm⁻¹, 349 (±2) cm⁻¹ or 1620 (±2) cm⁻¹, in particular comprising at least two peaks at positions 114 (±2) cm⁻¹, 132 (±2) cm⁻¹, 167 (±2) cm⁻¹, 349 (±2) cm⁻¹ or 1620 (±2) cm⁻¹, more particularly comprising the peaks at positions 114 (±2) cm⁻¹, 132 (±2) cm⁻¹, 167 (±2) cm⁻¹, 349 (±2) cm⁻¹ and 1620 (±2) cm⁻¹.
17. A solid form according to any one of aspects 1 to 15, further characterized by a Raman spectrum according to aspect 16.
18. A solid form according to any one of aspects 1 to 11, further characterized by a Raman spectrum comprising at the positions 1620 cm⁻¹ (±2) cm⁻¹.
19. A solid form according to any one of aspects 1 to 18, characterized by a raman spectrum as shown in FIG. 4.
20. A substantially pure solid form according to any one of aspects 1 to 19.
21. A solid form according to any one of aspects 1 to 20 for use as a medicament.
22. A solid form according to any one of aspects 1 to 20 for the treatment or prophylaxis of cancer, in particular BRAF associated cancer.
23. A solid form according to any one of aspects 1 to 20 for the treatment or prophylaxis of melanoma or colorectal cancer, in particular colorectal cancer.
24. A pharmaceutical composition comprising a solid form according to any one of aspects 1 to 20 and one or more pharmaceutically acceptable auxiliary substances.
25. Use of a solid form according to any one of aspects 1 to 20 for the treatment of cancer, in particular BRAF associated cancer.
26. Use of a solid form according to any one of aspects 1 to 20 for the treatment of melanoma or colorectal cancer, in particular colorectal cancer.
27. Use of a solid form according to any one of aspects 1 to 20 for the preparation of a medicament for the treatment of cancer, in particular BRAF associated cancer.
28. A method for therapeutic or prophylactic treatment of cancer, in particular BRAF associated cancer, said method comprising administering an effective amount of a solid form according to any one of aspects 1 to 20 to a patient in need thereof.

In one embodiment, the crystalline polymorphic Form B of compound of formula (I) is anhydrous, i.e. free of water bound in the crystal lattice, and non-hygroscopic (<0.2% water uptake according to European Pharmacopeia).

The invention also relates to a compound according to the invention when manufactured according to a process of the invention.

### Pharmaceutical Compositions

The compound of formula (I) in its various solid forms can be used as therapeutically active substance, e.g. in the form of a pharmaceutical composition. The pharmaceutical composition can be administered orally, e.g. in the form of tablets, coated tablets, dragées, hard and soft gelatin capsules, solutions, emulsions or suspensions. The administration can, however, also be effected rectally, e.g. in the form of suppositories, or parenterally, e.g. in the form of injection solutions.

The compound of formula (I) can be processed with a pharmaceutically inert, inorganic or organic carriers for the production of a pharmaceutical composition. Lactose, corn starch or derivatives thereof, talc, stearic acids or its salts and the like can be used, for example, as such carriers for tablets, coated tablets, dragées and hard gelatin capsules. Suitable carriers for soft gelatin capsules are, for example, vegetable oils, waxes, fats, semi-solid and liquid polyols and the like. Depending on the nature of the active substance no carriers are however usually required in the case of soft gelatin capsules. Suitable carriers for the production of solutions and syrups are, for example, water, polyols, glycerol, vegetable oil and the like. Suitable carriers for suppositories are, for example, natural or hardened oils, waxes, fats, semi-liquid or liquid polyols and the like.

The pharmaceutical composition can, moreover, contain pharmaceutically acceptable auxiliary substances such as preservatives, solubilizers, stabilizers, wetting agents, emulsifiers, sweeteners, colorants, flavorants, salts for varying the osmotic pressure, buffers, masking agents or antioxidants. They can also contain still other therapeutically valuable substances.

Pharmaceutical compositions comprising a compound of formula (I) alone or in combination, can be prepared for storage by mixing the active ingredient having the desired degree of purity with optional pharmaceutically acceptable carriers, excipients or stabilizers (Remington's Pharmaceutical Sciences 16th edition, Osol, A. (ed.) (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

Medicaments containing the a solid form of the compound of formula (I) as described herein and a therapeutically inert carrier are also provided by the present invention, as is a process for their production, which comprises bringing one or more compounds of formula (I) and/or pharmaceutically acceptable solvates thereof and, if desired, one or more other therapeutically valuable substances into a galenical administration form together with one or more therapeutically inert carriers.

Pharmaceutical compositions of a BRAF inhibitor include those suitable for oral, nasal, topical (including buccal and sublingual), rectal, vaginal and/or parenteral administration.

The dosage can vary within wide limits and will, of course, have to be adjusted to the individual requirements in each particular case. In the case of oral administration the dosage for adults can vary from about 200 mg to about 4000 mg per day of a compound of general formula (I) or of the corresponding amount of a pharmaceutically acceptable salt or a pharmaceutically acceptable solvate thereof. The daily dosage may be administered as single dose or in divided doses and, in addition, the upper limit can also be exceeded when this is found to be indicated.

The following examples illustrate the present invention without limiting it, but serve merely as representative thereof. The pharmaceutical preparations conveniently contain about 5-500 mg, particularly 100-500 mg, of a compound of formula (I). Examples of compositions according to the invention are:

### Example A

Tablets of the following composition are manufactured in the usual manner:

TABLE 1
possible tablet composition

mg/tablet

| ingredient | 5 | 25 | 100 | 500 |
|---|---|---|---|---|
| Compound of formula (I) | 5 | 25 | 100 | 500 |
| Lactose Anhydrous DTG | 125 | 105 | 30 | 150 |
| Sta-Rx 1500 | 6 | 6 | 6 | 60 |
| Microcrystalline Cellulose | 30 | 30 | 30 | 450 |
| Magnesium Stearate | 1 | 1 | 1 | 1 |
| Total | 167 | 167 | 167 | 831 |

### Manufacturing Procedure

1. Mix ingredients 1, 2, 3 and 4 and granulate with purified water.
2. Dry the granules at 50° C.
3. Pass the granules through suitable milling equipment.
4. Add ingredient 5 and mix for three minutes; compress on a suitable press.

### Example B-1

Capsules of the following composition are manufactured:

TABLE 2
possible capsule ingredient composition

mg/capsule

| ingredient | 5 | 25 | 100 | 500 |
|---|---|---|---|---|
| Compound of formula (I) | 5 | 25 | 100 | 500 |
| Hydrous Lactose | 159 | 123 | 148 | — |
| Corn Starch | 25 | 35 | 40 | 70 |
| Talk | 10 | 15 | 10 | 25 |
| Magnesium Stearate | 1 | 2 | 2 | 5 |
| Total | 200 | 200 | 300 | 600 |

### Manufacturing Procedure

1. Mix ingredients 1, 2 and 3 in a suitable mixer for 30 minutes.
2. Add ingredients 4 and 5 and mix for 3 minutes.
3. Fill into a suitable capsule.

The compound of formula (I), lactose and corn starch are firstly mixed in a mixer and then in a comminuting machine. The mixture is returned to the mixer; the talc is added thereto and mixed thoroughly. The mixture is filled by machine into suitable capsules, e.g. hard gelatin capsules.

### Example B-2

Soft Gelatin Capsules of the following composition are manufactured:

TABLE 3
possible soft gelatin capsule ingredient composition

| ingredient | mg/capsule |
|---|---|
| Compound of formula (I) | 5 |
| Yellow wax | 8 |
| Hydrogenated Soya bean oil | 8 |
| Partially hydrogenated plant oils | 34 |
| Soya bean oil | 110 |
| Total | 165 |

TABLE 4
possible soft gelatin capsule composition

| ingredient | mg/capsule |
|---|---|
| Gelatin | 75 |
| Glycerol 85% | 32 |
| Karion 83 | 8 (dry matter) |
| Titan dioxide | 0.4 |
| Iron oxide yellow | 1.1 |
| Total | 116.5 |

### Manufacturing Procedure

The compound of formula (I) is dissolved in a warm melting of the other ingredients and the mixture is filled into soft gelatin capsules of appropriate size. The filled soft gelatin capsules are treated according to the usual procedures.

### Example C

Suppositories of the following composition are manufactured:

TABLE 5
possible suppository composition

| ingredient | mg/supp. |
|---|---|
| Compound of formula (I) | 15 |
| Suppository mass | 1285 |
| Total | 1300 |

### Manufacturing Procedure

The suppository mass is melted in a glass or steel vessel, mixed thoroughly and cooled to 45° C. Thereupon, the finely powdered compound of formula (I) is added thereto and stirred until it has dispersed completely. The mixture is poured into suppository moulds of suitable size, left to cool; the suppositories are then removed from the moulds and packed individually in wax paper or metal foil.

### Example D

Injection solutions of the following composition are manufactured:

TABLE 6
possible injection solution composition

| ingredient | mg/injection solution. |
|---|---|
| Compound of formula (I) | 3 |
| Polyethylene Glycol 400 | 150 |
| acetic acid | q.s. ad pH 5.0 |
| water for injection solutions | ad 1.0 ml |

### Manufacturing Procedure

The compound of formula (I) is dissolved in a mixture of Polyethylene Glycol 400 and water for injection (part). The pH is adjusted to 5.0 by acetic acid. The volume is adjusted to 1.0 ml by addition of the residual amount of water. The solution is filtered, filled into vials using an appropriate overage and sterilized.

### Example E

Sachets of the following composition are manufactured:

TABLE 7
possible sachet composition

| ingredient | mg/sachet |
|---|---|
| Compound of formula (I) | 50 |
| Lactose, fine powder | 1015 |
| Microcrystalline cellulose (AVICEL PH 102) | 1400 |
| Sodium carboxymethyl cellulose | 14 |
| Polyvinylpyrrolidon K 30 | 10 |
| Magnesium stearate | 10 |
| Flavoring additives | 1 |
| Total | 2500 |

### Manufacturing Procedure

The compound of formula (I) is mixed with lactose, microcrystalline cellulose and sodium carboxymethyl cellulose and granulated with a mixture of polyvinylpyrrolidone in water. The granulate is mixed with magnesium stearate and the flavoring additives and filled into sachets.

### BRIEF DESCRIPTION OF THE DRAWINGS

FIG. 1 illustrates a X-ray powder diffraction pattern of the polymorphic Form B of (3R)—N-[2-cyano-4-fluoro-3-(3-methyl-4-oxo-quinazolin-6-yl)oxy-phenyl]-3-fluoro-pyrrolidine-1-sulfonamide.

FIG. 2 is a thermogram of the polymorphic form B of (3R)—N-[2-cyano-4-fluoro-3-(3-methyl-4-oxo-quinazolin-6-yl)oxy-phenyl]-3-fluoro-pyrrolidine-1-sulfonamide obtained by differential scanning calorimetry (DSC). A sharp melting signal was observed (onset 212.3° C., peak 214.7° C., enthalpy 109 J/g) and decomposition occurs after melting.

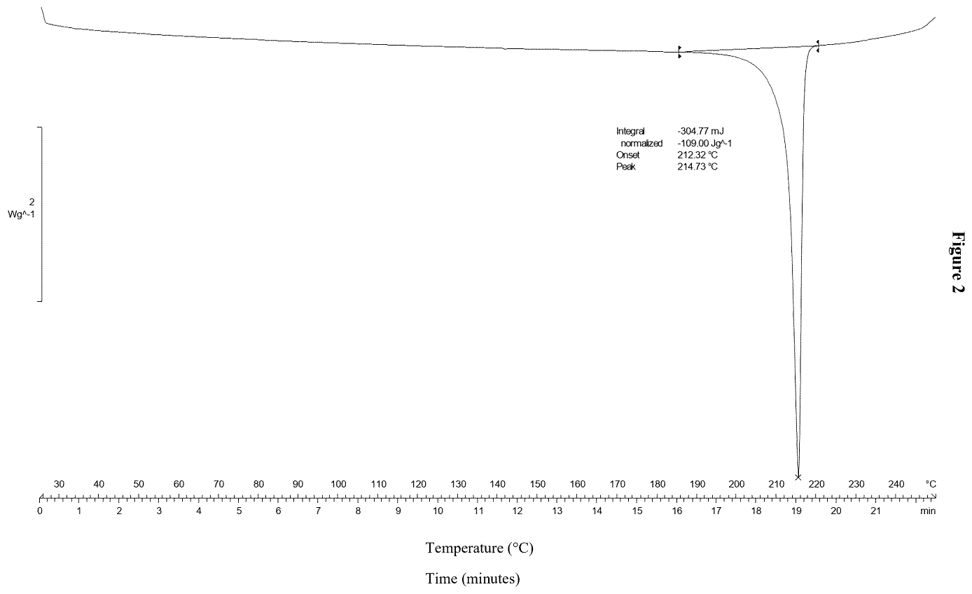

FIG. 3 is a thermogram of the polymorphic form B of (3R)—N-[2-cyano-4-fluoro-3-(3-methyl-4-oxo-quinazolin-6-yl)oxy-phenyl]-3-fluoro-pyrrolidine-1-sulfonamide obtained by thermogravimetric analysis (TGA). No significant massloss was observed (0.087%).

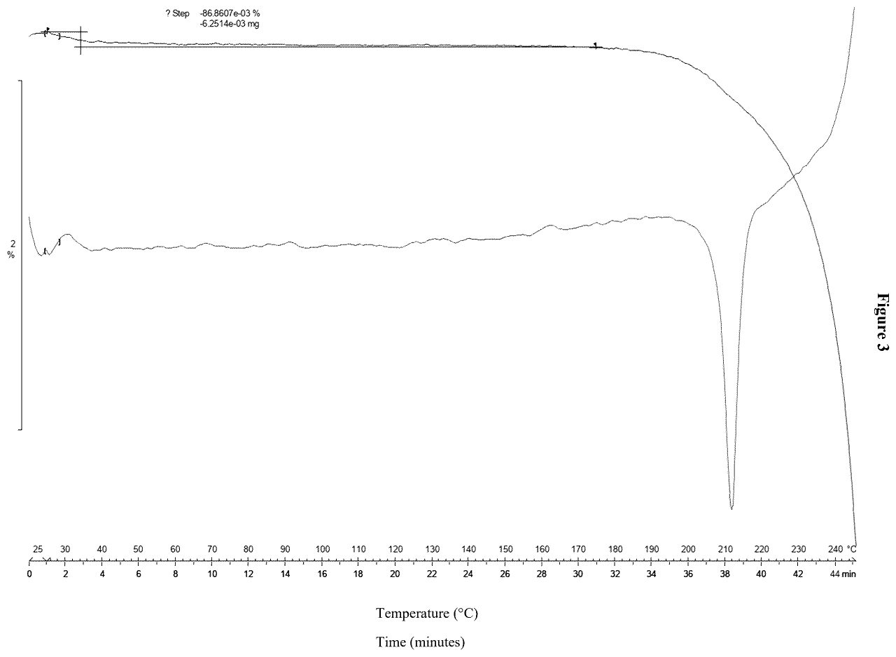

FIG. 4 is a raman spectrum of the polymorphic form B of (3R)—N-[2-cyano-4-fluoro-3-(3-methyl-4-oxo-quinazolin-6-yl)oxy-phenyl]-3-fluoro-pyrrolidine-1-sulfonamide.

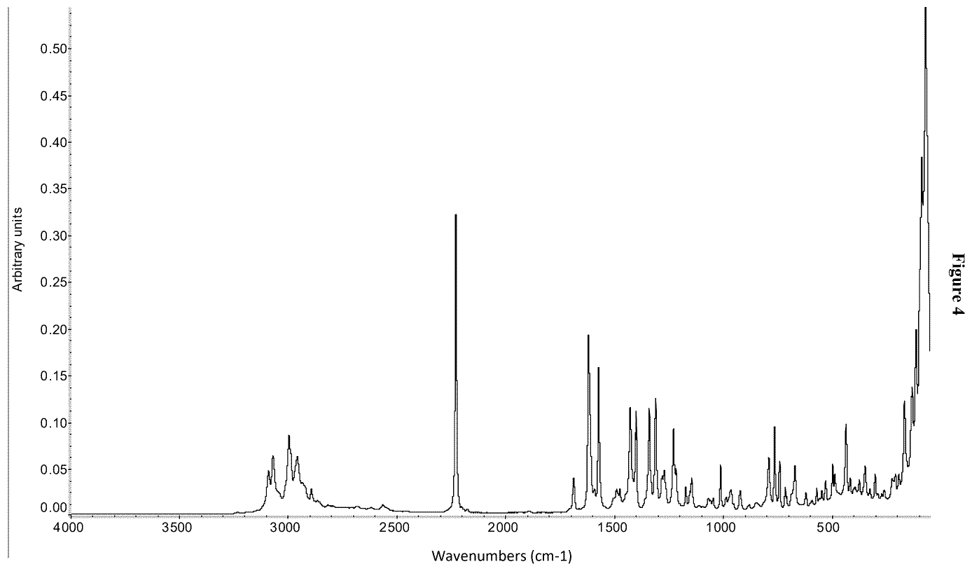

FIG. 5 is a IR spectrum of the polymorphic form B of (3R)—N-[2-cyano-4-fluoro-3-(3-methyl-4-oxo-quinazolin-6-yl)oxy-phenyl]-3-fluoro-pyrrolidine-1-sulfonamide.

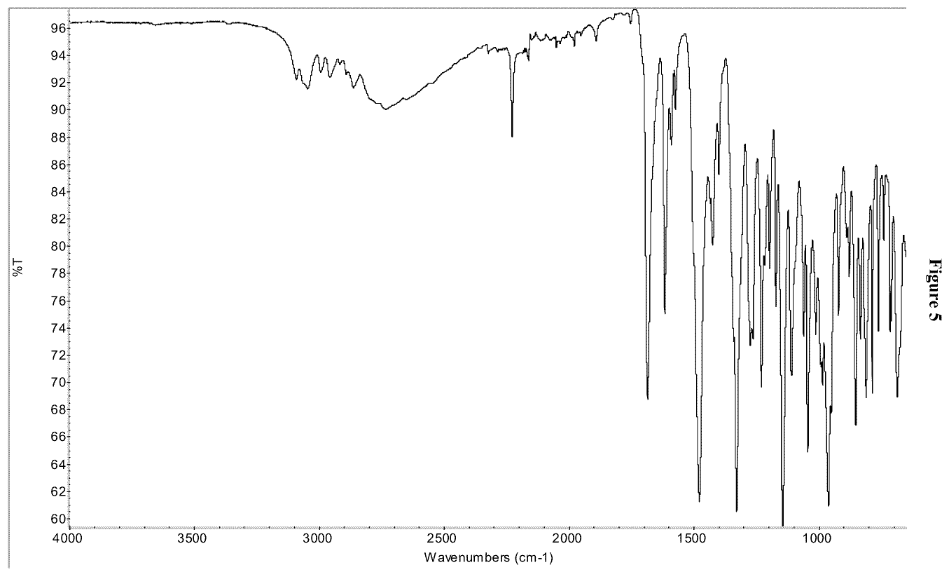

### EXPERIMENTAL PART

The following experiments are provided for illustration of the invention. They should not be considered as limiting the scope of the invention, but merely as being representative thereof.

### Abbreviations

ATR=attenuated total reflection; DCM=dichloromethane; DIPEA=N,N-diisopropylethylamine; DMF=dimethylformamide; DMSO dimethyl sulfoxide; DSC=differential scanning calorimetry; DVS=dynamic vapour sorption; ESI=electrospray ionization; EtOAc=ethyl acetate; FT=fourier transform; FTIR=fourier-transform infrared; IR=infrared; LC-MS/MS=liquid chromatography-MS/MS; MeOH=methanol; MS=mass spectrometry; RH=relative humidity; rt=room temperature; SFC=supercritical fluid chromatography; TGA=thermogravimetry.

### High Resolution X-Ray Powder Diffraction

High resolution X-ray powder diffraction (XRPD) patterns were recorded either in transmission geometry. X-ray diffraction patterns were recorded on a STOE STADI P diffractometer with CuKa1 radiation (1.5406 Å) and a Mythen position sensitive detector. The samples (approximately 10 to 50 mg) were prepared between thin polymer films and were usually analyzed without further processing (e.g., grinding or sieving) of the substance.

For polymorphic Form B the following peaks have been found by XRPD (expressed in values of degrees 2-theta) at approximately: 8.38, 9.76, 10.44, 10.62, 12.74, 12.88, 15.96, 16.68, 16.82, 17.20, 17.54, 18.76, 19.00, 19.40, 19.52, 20.04, 20.98, 21.24, 23.02, 23.30, 23.78, 24.02, 25.62, 25.88, 26.10, 26.48, 26.90, 27.24, 27.46, 28.56, 28.72, 28.94, 29.08, 29.28, 29.80, 30.22.

### Differential Scanning Calorimetry (DSC)

DSC curves were recorded using a Mettler-Toledo™ differential scanning calorimeter DSC2. System suitability tests were performed with Indium as reference substance and calibrations were carried out using Indium, Benzoic acid, Biphenyl and Zinc as reference substances.

For the measurements, approximately 2 to 6 mg (about 2.796 mg of Form B) of sample were placed in aluminum pans, accurately weighed and hermetically closed with perforation lids. Prior to measurement, the lids were pierced resulting in approx. 0.5 mm pin holes. The samples were then heated under a flow of nitrogen of about 100 mL/min using heating rates of typically 1 to 20, usually 10 K/min to a maximum temperature of typically 180° C. to 350° C. depending on decomposition temperature.

### Thermogravimetry (TGA)

Thermogravimetric analyses (TGA) were performed on a Mettler-Toledo™ thermogravimetric analyzer (TGA/DSC1 or TGA/DSC3+). System suitability tests were performed with Hydranal as reference substance and calibrations using Aluminum and Indium as reference substances.

For the thermogravimetric analyses, approx. 5 to 15 mg (about 7.197 mg of Form B) of sample were placed in aluminum pans, accurately weighed and hermetically closed with perforation lids. Prior to measurement, the lids were automatically pierced resulting in approx. 0.5 mm pin holes. The samples were then heated under a flow of nitrogen of about 50 mL/min using a heating rate of 5 K/min to a maximum temperature of typically 350° C.

### Moisture Sorption/Desorption

Moisture sorption/desorption data was collected on a DVS Advantage, a DVS Adventure, or a DVS Intrinsic (SMS Surface Measurements Systems) moisture balance system. The sorption/desorption isotherms were measured stepwise in a range from 0%-RH to 90%-RH at typically 25° C. A weight change of typically <0.001%/min was chosen as criterion to switch to the next level of relative humidity (with a maximum equilibration time of typically 24 hours, if the weight change criterion was not met). The data were corrected for the initial moisture content of the samples by taking the weight after drying of the samples at 0%-RH as zero point.

The hygroscopicity of a given substance was characterized (by close analogy with the European Pharmacopoeia) by the increase in mass when the relative humidity was raised from 0%-RH to 90%-RH:

| | |
|---|---|
| non-hygroscopic: | weight increase Dm < 0.2% |
| slightly hygroscopic: | weight increase 0.2% ≤ Dm < 2.0% |
| hygroscopic: | weight increase 2.0% ≤ Dm < 15.0% |
| very hygroscopic: | weight increase Dm ≥ 15.0% |
| deliquescent: | sufficient liquid is adsorbed to form a liquid |

European Pharmacopoeia-8″ Edition (2014), Chapter 5.11.

### IR Spectroscopy

The ATR FTIR spectra were recorded without any sample preparation using a ThermoNicolet iS5 FTIR spectrometer with ATR accessory. The spectral range was between 4000 cm⁻¹ and 650 cm⁻¹, resolution 2 cm⁻¹, and at least 50 co-added scans were collected. Happ-Genzel apodization was applied. Using ATR FTIR will cause the relative intensities of infrared bands to differ from those seen in a transmission FTIR spectrum using KBr disc or nujol mull sample preparations. Due to the nature of ATR FTIR, the bands at lower wavenumber are more intense than those at higher wavenumber.

Peakpicking was performed using Thermo Scientific Omnic 8.3 software using the automated ‘Find Peaks’ function. The ‘threshold’ and ‘sensitivity’ were manually adjusted to get a representative number of peaks.

TABLE 8
list of peaks identified by infrared
spectroscopy of polymorphic Form B.

686
709
714
740
762
786
811
832
852
878
888
921
950
961
985
991
1012
1044
1060
1109
1145
1172
1197
1217
1229
1263
1274
1329
1400
1425
1478
1573
1590
1617
1685
1891
2227
2733
2864
2957
2995
3047
3092

### Raman Spectroscopy

The FT-Raman spectra were recorded without any sample preparation in the spectral range of 4000-50 cm⁻¹ with a Bruker MultiRam FT-Raman spectrometer, equipped with a NdYAG 1064 nm laser and a liquid nitrogen cooled Germanium detector. The laser power at the sample was about 300 mW, 2 cm⁻¹ resolution was used, and 2048 scans were co-added. The Blackman-Harris 4-term apodization function was used. About 5 mg of sample (powder in a glass vial) were needed. Peakpicking was performed using Thermo Scientific Omnic 8.3 software using the automated ‘Find Peaks’ function. The ‘threshold’ and ‘sensitivity’ were manually adjusted to get a representative number of peaks.

TABLE 9
list of peaks identified by Raman
spectroscopy of polymorphic Form B.

69
88
114
132
167
193
209
260
302
326
349
375
393
417
435
487
497
529
547
571
592
621
671
715
740
764
791
847
880
923
966
986
1013
1047
1069
1146
1172
1218
1228
1271
1282
1312
1341
1401
1428
1476
1491
1573
1592
1620
1688
2228
2564
2894
2958
2996
3071
3091

### Synthesis and Crysatallization Protocol

The synthesis of the active pharmaceutical ingredient (API) (3R)—N-[2-cyano-4-fluoro-3-(3-methyl-4-oxo-quinazolin-6-yl)oxy-phenyl]-3-fluoro-pyrrolidine-1-sulfonamide was previously disclosed in WO2021/116055 and WO2022/258584. Importantly, WO2022/258584 also described a procedure to isolate polymorphic solid form A and the amorphous form of (3R)—N-[2-cyano-4-fluoro-3-(3-methyl-4-oxo-quinazolin-6-yl)oxy-phenyl]-3-fluoro-pyrrolidine-1-sulfonamide.
Crystallization-Protocol I: 100 g API were suspended in 1.23 kg acetone and 0.39 kg water at 370-400 rpm. The suspension was heated to 40° C. until a clear solution was obtained. The pressure was decreased from 400 mbar to 350 mbar to distill until a volume of 450 mL (349.1 g) of condensate was collected. The suspension was heated to 55° C. and a suspension of Form A seeds (250 mg in 390.91 mg water and 1.23 g acetone) was added and cooled down to 40° C. while stirring within 10 minutes. The suspension was distilled at 45° C. and 340 mbar until 780 mL of distillate were collected. The suspension was cooled to 20-25° C. room temperature within 2 hours and stirred there for 0.5 hours. On the next day, the suspension was filtered and washed twice with a 1+1 (w/w) mixture of water and acetone (total 250 g). The obtained product was dried at 45° C. and 20-25 mbar. The material was analyzed by XRPD and surprisingly identified as new solid form that was subsequently called Form B.
Crystallization-(alternative) Protocol II: 20 mg of Form A have been dissolved in 2 mL acetone at 40° C. The slightly brownish solution was filtered hot through a 0.45 μm syringe filter (PTFE) into a clean 2 mL vial and immediately placed at −10° C. An off-white solid was separated from the solution after 17 hours and analyzed via XRPD without further drying, identifying it as Form B.

1. A solid form of a compound of formula (I) 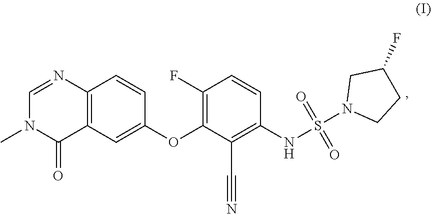 [SMILES: Cn1cnc2ccc(Oc3c(F)ccc(NS(=O)(=O)N4CC[C@@H](F)C4)c3C#N)cc2c1=O]
wherein the solid form is crystalline polymorphic Form B characterized by a X-ray powder diffraction pattern comprising a peak at an angle of diffraction at about 12.88 degrees 2-theta and at least one additional peak expressed in values of degrees 2-theta at about 10.62, 15.96, 16.82, 17.20, 20.04, 21.24, 23.78, 25.62, 25.88 or 26.90.

2. The solid form according to claim 1, characterized by an X-ray powder diffraction pattern comprising a peak at an angle of diffraction at about 12.88 degrees 2-theta and a peak at about 10.62 degrees 2-theta.

3. The solid form according to claim 1, characterized by an X-ray powder diffraction pattern comprising a peak at an angle of diffraction at about 12.88 degrees 2-theta and a peak at about 10.62 degrees 2-theta; wherein the pattern is further comprising at least one additional peak expressed in values of degrees 2-theta at about 15.96, 16.82, 17.20, 20.04, 21.24, 23.78, 25.62, 25.88 or 26.90.

4. The solid form according to claim 1, characterized by an X-ray powder diffraction pattern comprising at least three of the peaks at an angle of diffraction at about 10.62, 12.88, 16.82, 20.04 or 26.90 degrees 2-theta.

5. The solid form according to claim 1, characterized by an X-ray powder diffraction pattern comprising peaks at an angle of diffraction at about 10.62, 12.88, 15.96, 16.82, 17.20, 20.04, 21.24, 23.78, 25.62, 25.88 and 26.90 degrees 2-theta.

6. The solid form according to claim 1, which is further comprising a peak expressed in values of degrees 2-theta at about 8.38.

7. The solid form characterized by an X-Ray powder diffraction pattern according to claim 1, which is further comprising at least one additional peak expressed in values of degrees 2-theta at about 9.76, 10.44, 12.74, 16.68, 17.54, 18.76, 19.00, 19.40, 19.52, 20.98, 23.02, 23.30, 24.02, 26.10, 26.48, 27.24, 27.46, 28.56, 28.72, 28.94, 29.08, 29.28, 29.80 or 30.22.

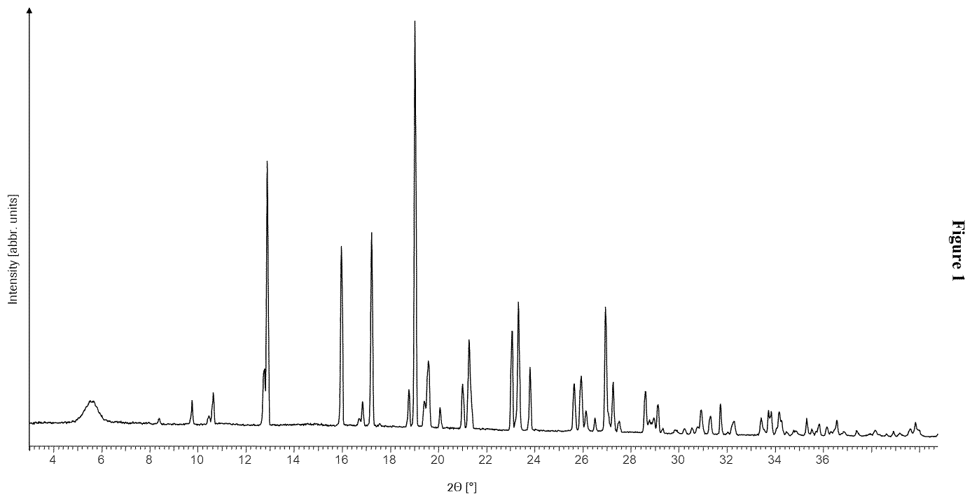

8. The solid form according to claim 1, characterized by an X-ray powder diffraction pattern as shown in FIG. 1.

9. The solid form according to claim 1, characterized by having a melting point with a peak signal at about 214.2° C. to about 215.2° C., in particular with a peak signal at about 214.7° C., using differential scanning calorimetry with a heating rate of 10 K/min.

10. A solid form of a compound of formula (I) 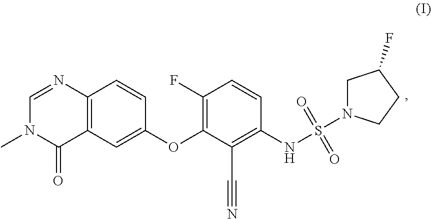 [SMILES: Cn1cnc2ccc(Oc3c(F)ccc(NS(=O)(=O)N4CC[C@@H](F)C4)c3C#N)cc2c1=O]
wherein the solid form is crystalline polymorphic Form B characterized by an IR spectrum comprising at least one peak at one of the positions 1685 cm⁻¹ (±2) cm⁻¹, 1617 cm⁻¹ (±2) cm⁻¹, 1425 cm⁻¹ (±2) cm⁻¹, 852 cm⁻¹ (±2) cm⁻¹ or 762 cm⁻¹ (±2) cm⁻¹, in particular comprising at least two peaks at positions 1685 cm⁻¹ (±2) cm⁻¹, 1617 cm⁻¹ (±2) cm⁻¹, 1425 cm⁻¹ (±2) cm⁻¹, 852 cm⁻¹ (±2) cm⁻¹ or 762 cm⁻¹ (±2) cm⁻¹, more particularly comprising the peaks at positions 1685 cm⁻¹ (±2) cm⁻¹, 1617 cm⁻¹ (±2) cm⁻¹, 1425 cm⁻¹ (±2) cm⁻¹, 852 cm⁻¹ (±2) cm⁻¹ and 762 cm⁻¹ (±2) cm⁻¹.

11. A solid form of a compound of formula (I) 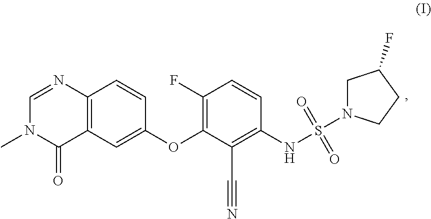 [SMILES: Cn1cnc2ccc(Oc3c(F)ccc(NS(=O)(=O)N4CC[C@@H](F)C4)c3C#N)cc2c1=O]
wherein the solid form is crystalline polymorphic Form B characterized by a Raman spectrum comprising at least one peak at one of the positions 114 (±2) cm⁻¹, 132 (±2) cm⁻¹, 167 (±2) cm⁻¹, 349 (±2) cm⁻¹ or 1620 (=2) cm⁻¹, in particular comprising at least two peaks at positions 114 (±2) cm⁻¹, 132 (±2) cm⁻¹, 167 (±2) cm⁻¹, 349 (±2) cm⁻¹ or 1620 (±2) cm⁻¹, more particularly comprising the peaks at positions 114 (±2) cm⁻¹, 132 (±2) cm⁻¹, 167 (±2) cm⁻¹, 349 (±2) cm⁻¹ and 1620 (±2) cm⁻¹.

12. A substantially pure solid form according to claim 1.

13-15. (canceled)

16. A pharmaceutical composition comprising a solid form according to claim 1, and one or more pharmaceutically acceptable auxiliary substances.

17-18. (canceled)

19. A method for treatment of BRAF associated cancer, said method comprising administering an effective amount of a solid form according to claim 1 to a patient in need thereof.

20. The method of claim 19, wherein BRAF associated cancer is melanoma or colorectal cancer.
